(19) United States
(12) Patent Application Publication
(10) Pub. No.: US 2023/0338253 A1
(43) Pub. Date: Oct. 26, 2023

(54) COSMETICS

(71) Applicant: SHISEIDO COMPANY, LTD., Tokyo (JP)

(72) Inventors: Kosuke ARIMOTO, Tokyo (JP); Yoshiro SADAKAMI, Tokyo (JP)

(73) Assignee: SHISEIDO COMPANY, LTD., Tokyo (JP)

(21) Appl. No.: 18/012,257

(22) PCT Filed: Jul. 8, 2021
(86) PCT No.: PCT/JP2021/025825

§ 371 Date: Dec. 22, 2022

(30) Foreign Application Priority Data
Jul. 22, 2020 (JP) 2020-125112

(51) Int. Cl. A61K 8/36; A61K 8/06; A61K 8/25; A61K 8/49; A61Q 17/04

(52) U.S. Cl. CPC A61K 8/361; A61K 8/064; A61K 8/25; A61K 8/4973; A61Q 17/04; A61K 2800/43; A61K 2800/41

(57) ABSTRACT

[Technical problem] To suppress the occurrence of color streaks in a cosmetic comprising two or more kinds of pigments having different average particle diameters.

[Solution] A cosmetic comprises at least two kinds of pigments having different average particle diameters. The cosmetic preparation has a lightness L* defined by CIE 1976 L*a*b* color system of 65 or less. The lightness L* is determined by filling a borosilicate glass container having a shell diameter φ of 35 mm with the cosmetic preparation to a height of 10 mm, and measuring according to an SCI method from the bottom surface of the filled glass container with use of an illuminant C. The cospmetic comprises behenic acid or a derivative thereof, wherein the blending amount is 0.15 to 2% by mass based on the total amount of the cosmetic.

### TECHNICAL FIELD

The present invention relates to a cosmetic, and particularly to a low-lightness cosmetic comprising a pigment.

### BACKGROUND ART

Conventionally, by blending an inorganic powder in a cosmetic, functions such as protection of the skin from ultraviolet rays, concealment of stains, freckles and the like, sebum absorption, and the like are imparted. The inorganic powder is hardly dispersed in an oily base, and in the case of an emulsified cosmetic, when a surfactant is added to an oil phase containing the powder, and an aqueous phase is added to cause emulsification, aggregation may occur due to the interaction between the powder, the surfactant, and water. In order to solve such a problem, the applicant has proposed a powder cosmetic in which the powder is hardly aggregated in a low viscosity region (Patent Literature 1).

In cosmetics, particularly cosmetics such as a foundation and the like, a pigment is blended as a so-called coloring material, in addition to the inorganic powders as described above (for example, Patent Literature 2). A plurality of types of pigments are generally blended in order to prepare a subtle color, and in order to facilitate visual recognition of the subtle color, pigments are often put in a transparent container and distributed on the market. In recent years, there is a demand for using dark foundations according to the season or a use scene.

### CITATION LIST

### Patent Literature

Patent Literature 1: JP 2013-256450 A

Patent Literature 2: JP 2018-35092 A

### SUMMARY OF THE INVENTION

### Object of the Invention

It has been found that when a cosmetic is prepared using a plurality of types of dark pigments according to such requirements, color streaks are generated due to stirring during preparation. This color streak is an important problem because it has a poor appearance when distributed in a transparent container, and there is a concern that it may be regarded as a quality trouble by consumers. In addition, since a gel-like cosmetic or the like is likely to cause a phenomenon in which moisture retained between polymers is separated, that is, so-called syneresis, it is preferable to further prevent syneresis and improve stability.

That is, an object of the present invention is to provide a cosmetic capable of preventing occurrence of color streaks and syneresis in a cosmetic comprising two or more kinds of pigments having different average particle diameters and having a dark color, in other words, a low lightness.

### Solution to Problem

The cosmetic according to the present invention is a cosmetic comprising:
(A) at least two kinds of pigments having different average particle diameters and
(B) behenic acid or a derivative thereof, wherein the amount of the (B) behenic acid or derivative thereof is 0.15 to 2% by mass based on the total amount of the cosmetic, and the lightness L* of the cosmetic is 65 or less, wherein the lightness L* is defined by CIE 1976 L*a*b* color system as determined by filling a borosilicate glass container having a shell diameter cp of 35 mm with the cosmetic to a height of 10 mm, and measuring according to an SCI method from the bottom surface of the glass container with use of an illuminant C is 65 or less.

The cosmetic according to the present invention preferably further comprises (C) a surfactant.

The (C) surfactant is preferably a sorbitan derivative.

The sorbitan derivative is preferably sorbitan sesquiisostearate.

The cosmetic according to the present invention preferably further comprises (D) a volatile linear silicone.

The cosmetic according to the present invention is preferably of a water-in-oil type.

### Advantageous Effects of the Invention

Since the cosmetic according to the present invention is a cosmetic comprising:
(A) at least two kinds of pigments having different average particle diameters and
(B) behenic acid or a derivative thereof, wherein
the amount of the (B)behenic acid or derivative thereof is 0.15 to 2% by mass based on the total amount of the cosmetic,
even if the lightness L* of the cosmetic is 65 or less. The lightness L* is defined by CIE 1976 L*a*b* color system as determined by filling a borosilicate glass container having a shell diameter φ of 35 mm with the cosmetic to a height of 10 mm, and measuring according to an SCI method from the bottom surface of the glass container with use of an illuminant C. It is possible to prevent the classification of the pigment due to the difference in the average particle diameter of the pigment, and to prevent the occurrence of color streaks and syneresis.

### BEST MODE FOR CARRYING OUT THE INVENTION

The cosmetic according to the present invention is a cosmetic comprising:
(A) at least two kinds of pigments having different average particle diameters and
(B) behenic acid or a derivative thereof, wherein
the amount of the (B) behenic acid or derivative thereof is 0.15 to 2% by mass based on the total amount of the cosmetic, and
the lightness L* of the cosmetic is 65 or less, wherein the lightness L* is defined by CIE 1976 L*a*b* color system as determined by filling a borosilicate glass container having a shell diameter φ of 35 mm with the cosmetic to a height of 10 mm, and measuring according to an SCI method from the bottom surface of the glass container with use of an illuminant C.

Hereinafter, each component will be described in detail. In the present specification, PEG stands for polyethylene glycol, POE stands for polyoxyethylene, and POP stands for polyoxypropylene.

(A) Pigment

The cosmetic of the present invention comprises at least two kinds of pigments having different average particle diameters. Here, the pigment is a pigment that colors an object without being dissolved in water, and is an inorganic pigment of a white pigment or a coloring pigment. More specific examples thereof include white pigments such as titanium dioxide (coloring is performed at an average particle diameter of 100 nm or more), zinc oxide, and the like, and coloring pigments such as red iron oxide, yellow iron oxide, black iron oxide, ultramarine blue, Prussian blue, manganese violet, carbon black, and the like. As the pigment, those that have been subjected to a surface treatment (hydrophobization treatment, hydrophilization treatment, water repellency treatment, and the like) as necessary may be used. The hydrophobization treatment includes preferably tetra hydrotetramethylcyclotetrasiloxane (EP-1 treatment), triethoxycaprylylsilane (OTS treatment) and the like.

The average particle diameter is measured by a laser diffraction scattering method (dry method) using a particle size distribution measuring apparatus (MT 3000) manufactured by Microtrac MRB, and is represented by a median diameter. The difference in average particle diameter means that two or more kinds of pigments contained in the cosmetic are different in average particle diameter to such an extent that classification occurs, and it is assumed that there is generally a difference of 0.1 to 2 μm in average particle diameter although it varies depending on the kind of the pigment.

The amount of the pigment is preferably more than 9.5% by mass and 30% by mass or less, more preferably 9.6 to 25% by mass, and still more preferably 10 to 20% by mass. When the content is more than 9.5% by mass, it becomes easier to prepare a subtle color tone by blending various kinds of pigments, and when the content is 30% by mass or less, usability can be further improved.

(B) Behenic Acid or Derivative Thereof

The cosmetic according to the present invention comprises behenic acid (behenic acid) or a derivative thereof. The derivative of behenic acid includes behenyl alcohol, behenic acid ester, behenic acid amide, behenic acid salt, behenic acid carbonate, behenic acid carbamate, behenic acid imide and/or behenic anhydride, and the like. The behenic acid ester includes preferably glyceryl behenate, and examples thereof include glyceryl (behenate/eicosanedioate) (NOMCORT HK-G (manufactured by The Nisshin OilliO Group, Ltd.) as a commercially available product), polyglyceryl-10 (behenate/eicosanedioate) (NOMCORT HK-P (manufactured by The Nisshin OilliO Group, Ltd.) as a commercially available product), and the like. The behenic acid or derivatives thereof may be used alone or in combination of two or more thereof.

(B) Behenic acid or a derivative thereof is contained in an amount of 0.15 to 2% by mass, preferably 0.2 to 0.8% by mass, and more preferably 0.25 to 0.7% by mass based on the total amount of the cosmetic. When the content of (B) behenic acid or a derivative thereof is 0.15% by mass or more based on the total amount of the cosmetic, the occurrence of color streaks can be prevented. In addition, when the content is 2% by mass or less, it is possible to achieve light usability that easily stretches to the skin.

(Lightness)

Regarding the lightness of the cosmetic of the present invention, the lightness L* of the cosmetic is 65 or less. The lightness L* is defined by CIE 1976 L*a*b* color system as determined by filling a borosilicate glass container having a shell diameter φ of 35 mm with the cosmetic to a height of 10 mm, and measuring according to an SCI (Specular Component Include) method from the bottom surface of the glass container with use of an illuminant C. When the lightness L* is 65 or less, a cosmetic having a dark color can be obtained. More preferably, the lightness L* is preferably 60 or less, and more preferably 55 or less.

(C) Surfactant

The cosmetic according to the present invention preferably further comprises a surfactant. By the surfactant, the dispersibility of the pigment can be improved. Among surfactants, a surfactant having an HLB of 7 or less is preferably used from the viewpoint of improving dispersibility.

Examples of such a surfactant include sorbitan derivatives, for example, fatty acid-modified sorbitan which is one of ester compounds in which sorbitan and a fatty acid are bonded (for example, sorbitan sesquiisostearate), polyglycerin fatty acid esters (for example, mono-cottonseed oil fatty acid glycerin, glyceryl monoerucate, glyceryl sesquioleate, glyceryl monostearate, glyceryl α,α′-oleate pyroglutamate, glyceryl monostearate malic acid, and the like), sorbitan fatty acid esters (for example, sorbitan monooleate, sorbitan monoisostearate, sorbitan monolaurate, sorbitan monopalmitate, sorbitan monostearate, sorbitan sesquioleate, sorbitan trioleate, diglycerol sorbitan penta-2-ethylhexylate, diglycerol sorbitan tetra-2-ethylhexylate and the like), glycerin fatty acid esters, sucrose fatty acid esters, diglycerin fatty acid esters, ethylene glycol fatty acid esters, propylene glycol fatty acid esters (for example, propylene glycol monostearate and the like), hydrogenated castor oil derivatives, polyglycerin alkyl ethers, and the like. These surfactants may be used singly or in combination of two or more kinds thereof.

(D) Volatile Linear Silicone

The cosmetic according to the present invention preferably further comprises (D) a volatile linear silicone. The volatile linear silicone oil is a silicone oil having a boiling point of 250° C. or lower at 1 atm (101.325 kPa). Examples of the volatile linear silicone oil include low molecular weight methylpolysiloxanes (1cS, 1.5cS, 2cS, etc.) such as octamethyltrisiloxane, decamethyltrisiloxane, dimethicone (dimethylpolysiloxane), and the like.

The amount of the volatile linear silicone oil is preferably 2 to 30% by mass, and more preferably 5 to 20% by mass based on the total amount of the cosmetic. When the content of the volatile linear silicone oil is 2% by mass or more based on the total amount of the cosmetic, the skin condition at the time of application and after application can be made smoother, and when the content is 30% by mass or less, the skin can have a more smooth feel of use without oily feeling.

The viscosity of the cosmetic of the present invention is preferably 5000 to 50000 mPa·s, more preferably 8000 to 45000 mPa·s, and still more preferably 10000 to 40000 mPa·s. When the viscosity is 50,000 mPa·s or less, elongation at the time of application is light, and better use feeling can be obtained. On the other hand, when the viscosity is 5000 mPa·s or more, more sufficient stability can be obtained. The viscosity referred to herein is measured by a BL type viscometer (VS-A type, manufactured by Shibaura system) at 30° C. under measurement conditions of rotor No. 4, 12 rpm, and 1 minute.

The cosmetic according to the present invention is appropriately blended as necessary with components usually used in cosmetic products, pharmaceuticals and the like, for example, oils, water, alcohols, thickeners, chelating agents, antioxidants, ultraviolet absorbers, moisturizers, fragrances, various medicinal ingredients, preservatives, pH adjusting agents, neutralizing agents, and the like.

Among the optional blending components described above, the oils include higher fatty acids such as lauric acid, myristic acid, palmitic acid, stearic acid and the like, waxes such as solid paraffin, beeswax, hydrogenated castor oil, carnauba wax, varico wax and the like, animal and vegetable oils and fats such as beef tallow, lard, mutton tallow, squalane, coconut oil, palm oil, palm kernel oil, soybean oil, olive oil, cottonseed oil, jojoba oil, castor oil, lanolin and the like, mineral oils such as liquid paraffin, petrolatum and the like, and synthetic oils such as trimethylpropane triisostearate, isopropyl myristate, glycerol tri-2-ethylhexanate, pentaerythritol tetra-2-ethylhexanate, POEPOP pentaerythritol ether, and the like.

The alcohols include higher alcohols such as linear alcohols such as lauryl alcohol, cetyl alcohol, stearyl alcohol, myristyl alcohol, oleyl alcohol and the like, and branched alcohols such as monostearyl glycerin ether, lanolin sarcol, cholesterol, phytosterol, isostearyl alcohol and the like, lower alcohols such as methanol, ethanol, propanol, isopropanol and the like, sitosterol, lanosterol, and the like.

The thickeners include water-soluble polymers such as alginic acid-based polymers such as sodium alginate, propylene glycol alginate and the like, vinyl-based polymers such as polyvinyl methyl ether, carboxyvinyl polymer and the like, POE-based polymers, POE-POP copolymer-based polymers, acrylic polymers such as sodium polyacrylate, polyacrylic acid amide and the like, and inorganic water-soluble polymers such as polyethyleneimine, cationic polymers, bentonite, aluminum magnesium silicate, laponite, hectorite, silicic anhydride, and the like.

The chelating agents include citramalic acid, agaric acid, glyceric acid, shikimic acid, hinokitiol, gallic acid, tannic acid, caffeic acid, ethylenediaminetetraacetic acid, ethyleneglycoldiamine tetraacetic acid, diethylenetriaminepentaacetic acid, phytic acid, polyphosphoric acid, metaphosphoric acid, analogs thereof, and alkali metal salts and carboxylic acid esters thereof.

The ultraviolet absorbers include benzoic acid-based ultraviolet absorbers such as para-aminobenzoic acid and the like; anthranilic acid-based ultraviolet absorbers such as methyl anthranilate and the like; salicylic acid-based ultraviolet absorbers such as octyl salicylate and the like; cinnamic acid-based ultraviolet absorbers such as isopropyl para-methoxycinnamate, octyl para-methoxycinnamate; pyridazine derivatives such as dimorpholinopyridazinone and the like; benzoylmethane derivatives such as 4-methoxy-4′-t-butyldibenzoylmethane and the like; camphor derivatives such as 3-(4′-methylbenzylidene)-d,l-camphor, 3-benzylidene-d,l-camphor and the like; benzotriazole derivatives such as 2,2′-hydroxy-5-methylphenylbenzotriazole, 2-(2′-hydroxy-5′-t-octylphenyl)benzotriazole, 2-(2′-hydroxy-5′-methylphenylbenzotriazole and the like; and others: 2-phenyl-5-methylbenzoxazole; dibenzalazine; dianisoylmethane; 5-(3,3-dimethyl-2-norbornylidene)-3-pentane-2-one, and the like.

The moisturizers include polyethylene glycol, propylene glycol, dipropylene glycol, 1,3-butylene glycol, glycerin, diglycerin, xylitol, maltitol, maltose, D-mannitol, glucose, fructose, sodium chondroitin sulfate, sodium hyaluronate, sodium lactate, glucosamine, cyclodextrin, and the like.

The medicinal ingredients include vitamins such as vitamin A oil, retinol, retinol palmitate, pyridoxine hydrochloride, benzyl nicotinate, nicotinamide, dl-α-tocopherol nicotinate, magnesium ascorbate phosphate, vitamin D2, dl-α-tocopherol, pantothenic acid, biotin and the like; anti-inflammatory agents such as azulene, glycyrrhizin and the like; whitening agents such as arbutin and the like, hormones such as estradiol and the like; astringents such as zinc oxide, tannic acid and the like; refreshing agents such as L-menthol, camphor and the like; and others: lysozyme chloride, pyridoxine hydrochloride, sulfur, and the like can be blended. Furthermore, various extracts exhibiting various medicinal effects can be blended. That is, Houttuynia cordata extract, Phellodendron bark extract, Glycyrrhiza extract, Paeonia lactiflora extract, moutan bark extract, Luffa cylindrica extract, Saxifraga stolonifera extract, Eucalyptus extract, clove extract, marronnier extract, Centaurea cyanus extract, seaweed extract, thyme extract, and the like are mentioned.

The preservatives include paraoxybenzoic acid esters such as methylparaben, ethylparaben, butylparaben and the like, benzoic acid, salicylic acid, sorbic acid, parachlorometacresol, hexachlorophene, benzalkonium chloride, chlorhexidine chloride, trichlorocarbanilide, photosensitive elements, phenoxyethanol, and the like.

The neutralizing agents include 2-amino-2-methyl-1-propanol, 2-amino-2-methyl-1,3-propanediol, potassium hydroxide, potassium hydroxide, triethanolamine, sodium carbonate, and the like.

The pH adjusting agents include lactic acid, citric acid, glycolic acid, succinic acid, tartaric acid, malic acid, sodium hydrogen carbonate, ammonium hydrogen carbonate, and the like.

The antioxidants include ascorbic acid, α-tocopherol, carotenoid, and the like.

The above components are examples, and are not limited thereto. In addition, these components can be appropriately combined and blended according to a formulation according to a desired form.

The cosmetic of the present invention can be in the form of water-in-oil (W/O), and is produced from the above components by a conventional method. Specific examples of the product form include milky or cream-like products such as an emulsion, a skin care cream, a makeup base, a hair cream, a liquid foundation, a cushion foundation, an eyeliner, a mascara, an eyeshadow, and the like.

### EXAMPLES

Next, the present invention will be described more specifically with reference to examples. The present invention is not limited at all by the following examples.

### Examples 1 to 3 and Comparative Examples 1 to 8

Water-in-oil type cosmetics were prepared by a conventional method according to the formulation shown in Table 1, and for the obtained cosmetics, the lightness L* defined in the CIE 1976 L*a*b* color system was measured under the following conditions. In addition, color streaks and syneresis were evaluated according to the following criteria.

The details of the product names and the like of the raw materials in the table are as follows. Here, pigment-grade titanium oxide, hydrophobized iron oxide (red), hydrophobized iron oxide (yellow), and hydrophobized iron oxide (black) in the table are pigments.

Lauryl PEG-9 polydimethylsiloxyethyl dimethicone: silicone SC0928SL (manufactured by Shin-Etsu Chemical Co., Ltd.)

PEG-10 dimethicone: Silicone SC 9450 N (manufactured by Shin-Etsu Chemical Co., Ltd.)

Diphenylsiloxyphenyl trimethicone: Silicon KF 56 (manufactured by Shin-Etsu Chemical Co., Ltd.)

Sorbitan sesquiisostearate: ESTEMOL 182 V (manufactured by The Nisshin OilliO Group, Inc.)

Dimethicone: Silicone KF-96A-2CS (manufactured by Shin-Etsu Silicone Co., Ltd.)

Disteardimonium hectorite: Benton 38 VCG (manufactured by Elementis Specialties)

Fine particle titanium oxide: (average particle diameter: 15 nm)

Pigment-grade titanium oxide: (average particle diameter: 250 nm)

Hydrophobized iron oxide (red): (average particle diameter: 660 nm)

Hydrophobized iron oxide (yellow): (average particle diameter: 400 nm)

Hydrophobized iron oxide (black): (average particle diameter: 1.6 μm)

PEG-20: Polyethylene glycol 1500 (manufactured by NOF CORPORATION)

<Measurement of Lightness>

A cosmetic of each example was filled in a borosilicate glass container (Maruemu Corporation, screw tube bottle 50 mL transparent No. 7) having a mouth inner diameter of φ 20.3 mm and a shell diameter of φ 35 mm to a height of 10 mm, and measurement was performed by an SCI method using an illuminant C from the bottom surface of the glass container. The lightness L* was 24 in each case.

<Color Streak>

A turbine-type stirring blade was attached to a three-one motor (manufactured by Shinto Chemical Co., Ltd.) for the cosmetic of each example, the stirring blade was installed so that the bottom surface of the stirring blade was in contact with the surface of the cosmetic of each example, the cosmetic was rotated at 12 rpm for 4 hours, and then the bulk was visually observed and evaluated according to the following criteria.

(Evaluation criteria)

A: No color streak

B: There are blurred color streaks in a narrow range, but there is no problem in the entire appearance.

C: There are blurred color streaks in a wide range.

D: There are clear color streaks.

<Syneresis>

The cosmetic of each example was allowed to stand at 50° C. for 4 weeks, and then the presence or absence of syneresis (oil floating) was visually observed and evaluated according to the following evaluation criteria.

(Evaluation Criteria)

A: No syneresis

B: A small amount of transparent liquid is observed on the surface.

C: An accumulated state of a transparent liquid is observed.

TABLE 1

| | | | | | Com. | Com. | Com. | Com. | Com. | Com. | Com. | Com. |
|---|---|---|---|---|---|---|---|---|---|---|---|---|
| | | Ex. 1 | Ex. 2 | Ex. 3 | Ex. 1 | Ex. 2 | Ex. 3 | Ex. 4 | Ex. 5 | Ex. 6 | Ex. 7 | Ex. 8 |
| lauryl PEG-9 | | 2 | 2 | 2 | 2 | 2 | 2 | 2 | 2 | 2 | 2 | 2 |
| polydimethylsiloxyethyl dimethicone | | | | | | | | | | | | |
| PEG-10 dimethicone | | 2 | 2 | 2 | 2 | 2 | 2 | 2 | 2 | 2 | 2 | 2 |
| diphenylsiloxyphenyl trimethicone | | 6 | 6 | 6 | 6 | 6 | 6 | 6 | 6 | 6 | 6 | 6 |
| sorbitan sesquiisostearate | | 0.25 | 0.25 | | | 0.25 | 0.25 | 0.25 | 0.25 | 0.25 | 0.25 | 0.25 |
| isostearic acid | | | | | | | 0.5 | 1 | 2 | | | |
| stearic acid | | | | | | | | | | 0.5 | | |
| palmitic acid | | | | | | | | | | | 0.5 | |
| behenic acid | | 0.5 | 0.3 | 0.5 | | | | | | | | |
| myristic acid | | | | | | | | | | | | 0.5 |
| dimethicone | | 21.25 | 21.45 | 21.5 | 22 | 21.75 | 21.25 | 20.75 | 19.75 | 21.25 | 21.25 | 21.25 |
| ethylhexyl methoxycinnamate | | 4.5 | 4.5 | 4.5 | 4.5 | 4.5 | 4.5 | 4.5 | 4.5 | 4.5 | 4.5 | 4.5 |
| disteardimonium hectorite | | 1.1 | 1.1 | 1.1 | 1.1 | 1.1 | 1.1 | 1.1 | 1.1 | 1.1 | 1.1 | 1.1 |
| fine particle titanium oxide | | 1.5 | 1.5 | 1.5 | 1.5 | 1.5 | 1.5 | 1.5 | 1.5 | 1.5 | 1.5 | 1.5 |
| pigment-grade titanium oxide | | 1.4 | 1.4 | 1.4 | 1.4 | 1.4 | 1.4 | 1.4 | 1.4 | 1.4 | 1.4 | 1.4 |
| hydrophobized iron oxide (red) | | 3.3 | 3.3 | 3.3 | 3.3 | 3.3 | 3.3 | 3.3 | 3.3 | 3.3 | 3.3 | 3.3 |
| hydrophobized iron oxide (yellow) | | 7.7 | 7.7 | 7.7 | 7.7 | 7.7 | 7.7 | 7.7 | 7.7 | 7.7 | 7.7 | 7.7 |
| hydrophobized iron oxide (black) | | 3 | 3 | 3 | 3 | 3 | 3 | 3 | 3 | 3 | 3 | 3 |
| hydrophobized synthetic phlogopite | | 3 | 3 | 3 | 3 | 3 | 3 | 3 | 3 | 3 | 3 | 3 |
| ion exchanged water | | 20 | 20 | 20 | 20 | 20 | 20 | 20 | 20 | 20 | 20 | 20 |
| grycerol | | 7.5 | 7.5 | 7.5 | 7.5 | 7.5 | 7.5 | 7.5 | 7.5 | 7.5 | 7.5 | 7.5 |
| dipropylene glycol | | 10 | 10 | 11 | 10 | 10 | 10 | 10 | 10 | 10 | 10 | 10 |
| PEG-20 | | 5 | 5 | 5 | 5 | 5 | 5 | 5 | 5 | 5 | 5 | 5 |
| Total | | 100 | 100 | 100 | 100 | 100 | 100 | 100 | 100 | 100 | 100 | 100 |
| evaluation | color streak | A | A | B | D | C | C | B | A | A | A | A |
| | syneresis | A | A | A | A | A | A | B | C | B | B | B |

As shown in Table 1, in examples, syneresis did not occur, and color streaks were prevented to such an extent that no problem occurred in appearance. On the other hand, in Comparative Example 1 in which sorbitan sesquiisostearate and behenic acid used in examples were removed, Comparative Example 2 in which behenic acid was removed, and Comparative Examples 3 and 4 in which isostearic acid was used instead of behenic acid, color streaks occurred, and a problem occurred in appearance. In Comparative Example 5 in which isostearic acid was highly blended instead of behenic acid, color streaks did not occur, but syneresis was remarkably observed. In Comparative Example 6 in which stearic acid was used in place of behenic acid, Comparative Example 7 in which palmitic acid was used in place of behenic acid, and Comparative Example 8 in which myristic acid was used in place of behenic acid, color streaks did not occur, but syneresis was observed, and the stability was deteriorated.

1. A cosmetic comprising:
(A) at least two kinds of pigments having different average particle diameters; and
(B) behenic acid or a derivative thereof,
wherein the amount of the (B) behenic acid or derivative thereof is 0.15 to 2% by mass based on the total amount of the cosmetic, and
the lightness L* of the cosmetic is 65 or less,
wherein the lightness L* is defined by CIE 1976 L*a*b* color system as determined by filling a borosilicate glass container having a shell diameter φ of 35 mm with the cosmetic to a height of 10 mm, and measuring according to an SCI method from the bottom surface of the glass container with use of an illuminant C.

2. The cosmetic according to claim 1, further comprising (C) a surfactant.

3. The cosmetic according to claim 2, wherein the (C) surfactant is a sorbitan derivative.

4. The cosmetic according to claim 3, wherein the sorbitan derivative is sorbitan sesquiisostearate.

5. The cosmetic according to claim 1, to further comprising (D) a volatile linear silicone.

6. The cosmetic according to claim 1, which is of a water-in-oil type.
